(19) United States
(12) Patent Application Publication
(10) Pub. No.: US 2015/0032202 A1
(43) Pub. Date: Jan. 29, 2015

(54) Drug-Eluting Stent and Method

(71) Applicant: New York University, New York, NY (US)

(72) Inventors: Omar Tanweer, Old Westbury, NY (US); Vittoria Flamini, New York, NY (US)

(21) Appl. No.: 14/444,251

(22) Filed: Jul. 28, 2014

Related U.S. Application Data
(60) Provisional application No. 61/858,636, filed on Jul. 26, 2013.

(51) Int. Cl. A61L 31/16; A61F 2/90

(52) U.S. Cl. CPC A61L 31/16; A61F 2/90
USPC 623/1.42

(57) ABSTRACT

A drug-eluting stent is disclosed, the stent having an outer cylindrical portion, an inner cylindrical portion disposed within and substantially coaxial with the outer cylindrical portion, a plurality of cross-links connecting the inner cylindrical portion to the outer cylindrical portion, and a first drug affixed to the inner and/or outer cylindrical portions. The stent may further comprise a second drug affixed to the inner and/or outer cylindrical portions. Methods are disclosed for use of a drug-eluting stent for treatment of a region of interest which is located away (downstream) from the location of the stent. Methods are disclosed for the use of the dual drug-eluting stent to provide therapy and adjunctive therapy protocols.

### CROSS-REFERENCE TO RELATED APPLICATION

This application claims priority to U.S. Provisional Application No. 61/858,636, filed on Jul. 26, 2013, now pending, the disclosure of which is incorporated herein by reference.

### FIELD OF THE DISCLOSURE

The disclosure relates to stents, and more particularly to stents configured to deliver drugs to a region of interest.

### BACKGROUND OF THE DISCLOSURE

Primary brain tumors, such as Glioblastoma multiforme (GBM) and anaplastic astrocytoma (AA) remain amongst the most challenging entities to treat in Neuro-oncology and Neurosurgery. Despite advancement in treatments, these cancers kill up to 15,000 Americans a year, with a median overall survival durations of only 12-15 months for GBM, and 3-4 years for AA. Currently, combinations of surgical resection, radiation therapy, and chemotherapy are used for control measures. Temozolomide (Temodar) is part of the first line chemotherapy and bevacizumab (Avastin) is considered for recurrence.

Many obstacles are present in developing an ideal therapeutic agent for these and other types of tumors. One main obstacle is the inability to provide a steady flow of drug through the difficult to penetrate blood brain barrier (BBB). Chemotherapy delivery intra-arterially after BBB disruption has been used and involves giving a bolus of Mannitol in an artery feeding the tumor to disrupt the BBB, followed by a bolus of Avastin. The limitation of this technique is the one-time delivery of the medication and required return for subsequent dosing.

Previous drug-eluting stents have been designed for localized use generally to prevent the occurrence of stenosis or restenosis. Although such stents may cause some amount of a downstream effect of the eluted drug, such coincidental use has only been detected up to a few millimeters distal to the stent. Such coincidental distal elution has been of little significance in therapeutic use.

Accordingly, there is a need for a method and device for providing sustained drug delivery to a region of interest (i.e., a tumor) without the requirement of repeated invasive catheterization.

### BRIEF SUMMARY OF THE DISCLOSURE

A drug-eluting stent is disclosed, which is suitable for use in providing therapeutic effect downstream from the location of the stent. As such, the stent is suitable for use to provide, for example, chemotherapeutic treatment into a tumor via a feeding blood vessel. Additionally, the embodiments of the presently disclosed stent may include two drugs which have an additive effect, such as providing a chemotherapeutic agent in combination with a blood-brain-barrier disruptor (“BBBD”) in order to enhance the ability of chemotherapy to a tumor across the blood-brain-barrier.

The disclosed stent comprises an outer cylindrical portion, an inner cylindrical portion disposed within and substantially coaxial with the outer cylindrical portion, a plurality of cross-links connecting the inner cylindrical portion to the outer cylindrical portion, and a first drug affixed to the inner and/or outer cylindrical portions. The stent may further comprise a second drug affixed to the inner and/or outer cylindrical portions. Methods are disclosed for use of a drug-eluting stent for treatment of a region of interest which is located away (downstream) from the location of the stent. Methods are disclosed for the use of the dual drug-eluting stent to provide therapy and adjunctive therapy protocols, including, for example, causing the first drug to preferentially reach a region of interest (e.g., a therapy target) before the second drug.

### DESCRIPTION OF THE DRAWINGS

For a fuller understanding of the nature and objects of the disclosure, reference should be made to the following detailed description taken in conjunction with the accompanying drawings, in which:

FIG. 1 is a cross-sectional view of a drug-eluting stent according to an embodiment of the present disclosure, wherein the cross-section is taken at a plane perpendicular to a primary longitudinal axis of the stent;

FIG. 2 is an oblique view of the drug-eluting stent of FIG. 1;

FIG. 3A depicts a cross-link according to an embodiment of the present disclosure wherein the pivot is a coil; and

FIG. 3B depicts a cross-link according to another embodiment of the present disclosure wherein the pivot is a elastomeric material;

FIG. 3C is a cross-link according to another embodiment of the present disclosure;

FIGS. 4A and B are front perspective views of a drug-eluting stent in a closed state according to an embodiment of the present disclosure;

FIGS. 5A and B are isometric views of a portion of the drug-eluting stent of FIGS. 4A and B;

FIGS. 6A and B are side views of a portion of the drug-eluting stent of FIGS. 4A and B;

FIGS. 7A and B are front perspective views of the drug-eluting stent of FIGS. 4A and B in an open state;

FIGS. 8A and B are isometric views of the drug-eluting stent of FIGS. 7A and B;

FIGS. 9A and B are side views of the drug-eluting stent of FIGS. 7A and B;

FIGS. 10A and B are isometric views of the inner cylindrical portion of the drug-eluting stent of FIGS. 7A and B;

FIGS. 11A and B are side views of the inner cylindrical portion of the drug-eluting stent of FIGS. 7A and B;

FIGS. 12A and B are isometric views of the outer cylindrical portion of the drug-eluting stent of FIGS. 7A and B; and

FIGS. 13A and B are side views of the outer cylindrical portion of the drug-eluting stent of FIGS. 7A and B.

### DETAILED DESCRIPTION OF THE DISCLOSURE

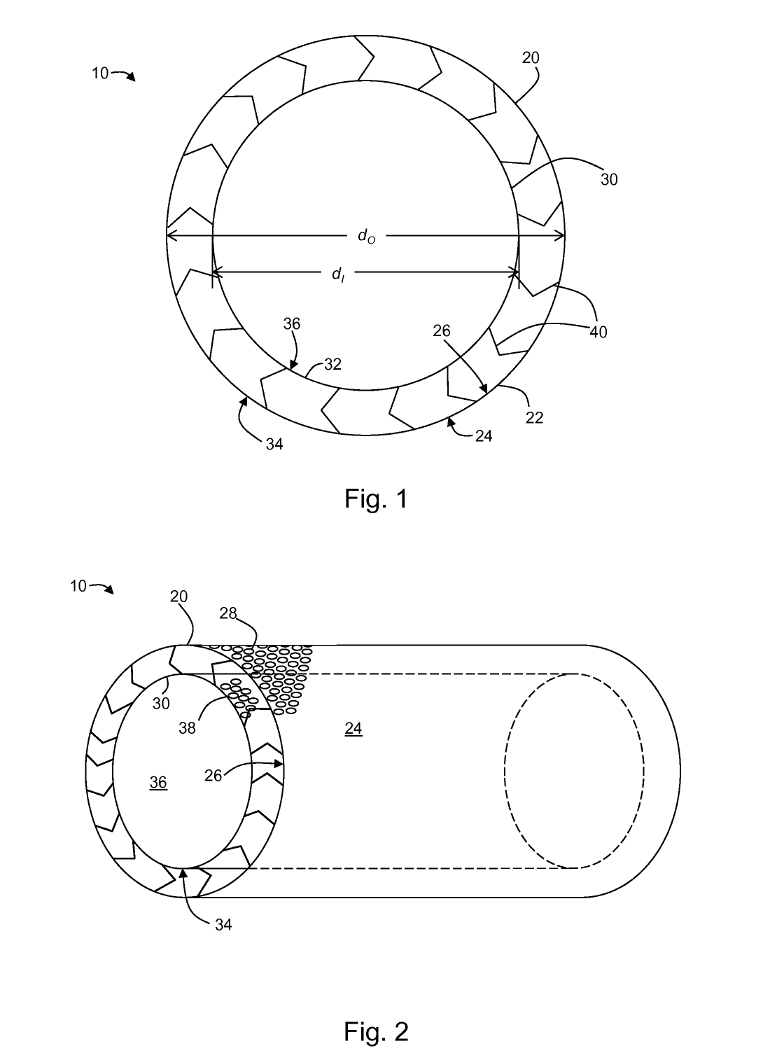

The present disclosure may be embodied as a drug-eluting stent 10, such as the stent 10 depicted in FIGS. 1 and 2. The stent 10 has an outer cylindrical portion 20, having an outer wall 22 defined by an abluminal surface 24 and a interlumen surface 26. A plurality of holes 28 are disposed in the outer wall 22. Each hole 28 extends from the abluminal surface 24 to the interlumen surface 26. The plurality of holes 28 define a porosity of the outer cylindrical portion 20, such that a greater number of holes 28 and/or holes 28 of a larger size yield a higher porosity.

The outer cylindrical portion 20 is configured to vary in diameter d_{O}. As such, the outer cylindrical portion 20 is expandable from an outer first diameter d_{O1}, for delivery into a vessel, to an outer second diameter d_{O2}, for placement at a location within the vessel.

The stent 10 further comprises an inner cylindrical portion 30, having an inner wall 32 defined by an interlumen surface 34 and a luminal surface 36. The inner cylindrical portion 30 is positioned within and substantially coaxial with the outer cylindrical portion 20. A plurality of holes 38 are disposed in the inner wall 32. Each hole 38 extends from the interlumen surface 34 to the luminal surface 36. The plurality of holes 38 define a porosity of the inner cylindrical portion 30, such that a greater number of holes 38 and/or holes 38 of a larger size yield a higher porosity. The porosity of the outer cylindrical portion 20 may be greater than, less than, or the equal to the porosity of the inner cylindrical portion 30.

The inner cylindrical portion 30 may be configured to vary in diameter d_{I} between a collapsed and an expanded state. As such, the inner cylindrical portion 30 may be expandable from an inner first diameter d_{I1}, for delivery into a vessel, to an inner second diameter d_{I2}, for placement at a location within the vessel.

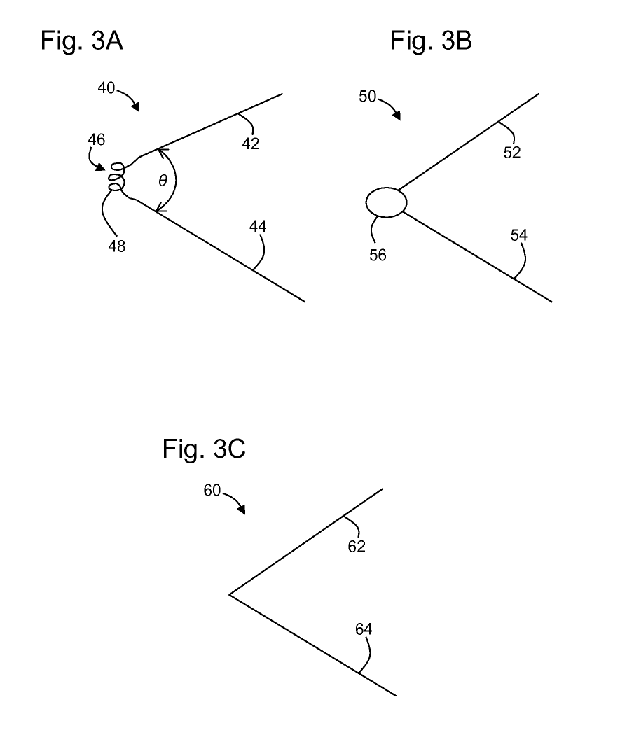

The stent 10 comprises a plurality of cross-links 40, which connect the outer cylindrical portion 20 to the inner cylindrical portion 30. The cross-links 40 are configured to allow a radial distance dᵣ between the outer cylindrical portion 20 and the inner cylindrical portion 30 to vary. In this way, the cross-links 40 are considered to be collapsible (and expandable). In some embodiments of cross-links 40 (see, e.g., FIG. 3A), each cross-link 40 comprises a first segment 42 and a second segment 44. The first and second segments 42, 44 may be connected by a pivot 46 such that the first and second segments 42, 44 are rotatable, relative to each other, about the pivot 46. The first and second segments 42, 44 form an angle θ. The pivot 46 may be biased to return to a desired angular relationship, θ, between the first and second segments when the cross-link 40 is in a relaxed state (i.e., not collapsed). θ may be any angle, for example, in embodiments of a cross-link 40, θ may be 10°, 30°, 45°, 60°, 90°, or any angle in the range of 1°-90°, inclusive. The pivot 46 may be a coil 48. In another embodiment (see, e.g., FIG. 3B), the cross-links 50 may comprise a first segment 52 and a second segment 54 connected by a pivot 56 made from a resilient material. In another embodiment of a cross-link 60 (see, e.g., FIG. 3C), the first segment 62 and second segment 64 are fixedly connected and the collapsibility of the cross-link 60 is effectuated by the ability of the first and/or second segments 62, 64 to bend.

The stent 10 further comprises a first drug disposed on at least one of the outer cylindrical portion 20 and inner cylindrical portion 30. For example, the first drug may be disposed on the abluminal surface 22 and/or the interlumen surface 24 of the outer cylindrical portion 20 and/or the first drug may be disposed on the luminal surface 34 and/or the interlumen surface 32 of the inner cylindrical portion 30. In some embodiments, the first drug is disposed in the plurality of holes 28 of the outer cylindrical portion 20 and/or the plurality of holes 38 of the inner cylindrical portion 30. In some embodiments, the first drug is disposed on the plurality of cross-links 40.

In some embodiments, the first drug is disposed on the outer cylindrical portion 20 of the stent 10 and the stent 10 further comprises a second drug disposed on the inner cylindrical portion 30. The dynamics of blood flow through the stent 10 will be defined in part by the diameter of each of the inner and outer cylindrical portions 20, 30. In some embodiments, blood flow through the lumen of the inner cylindrical portion 30 (the “central lumen 31”) will be at a higher velocity than blood flow through the inter-lumen space between the inner and outer cylindrical portions 20, 30. As such, it may be advantageous to select a drug for the luminal surface 36 of the inner cylindrical portion 30 that will act to increase the efficacy of a drug selected for the interlumen surfaces 22, 34. For example, a blood-brain-barrier disrupter (“BBBD”) may be disposed on the luminal surface such that the BBBD arrives at the target lesion prior to a chemotherapeutic agent disposed on one or more of the interlumen surfaces 22, 34. Other configurations will be apparent to one having skill in the art in light of the present disclosure. Additionally, because the blood flowing through the central lumen is closer to the center of the blood vessel, (i.e., radially further from the vessel wall), the blood flowing through the central lumen 31 will travel further downstream before coming into contact with the vessel wall thereby advantageously targeting distal regions.

The stent 10 may further comprise a second drug disposed on the first drug. As such, the first and second drugs are disposed on the outer cylindrical portion 20 and/or the inner cylindrical portion 30 in layers. The first and second drugs may be disposed on one another in two or more alternating layers. In this manner, when the stent 10 is deployed in a blood vessel, the drug of the outermost layer will elute first, and the drug of the adjacent layer will elute once such layer is exposed (which may occur in a localized fashion if the layers do not elute uniformly across and along the stent). Such a configuration is advantageous in treatment protocols wherein a first agent increases the efficacy of a second agent. For example, the second drug (in an outermost layer) may be a BBBD and the first drug may be a chemotherapeutic agent. In this example, the BBBD is eluted first to open the blood-brain barrier for the chemotherapeutic agent.

Suitable chemotherapeutic agents include, but are not limited to, the following:

Temazolomide (Temodar): an oral alkylating agent used against aggressive primary brain tumors (anaplastic astrocytoma and glioblastoma). This is a first line chemotherapeutic agent.

Bevacuziumab (Avastin): a humanized monoclonal antibody that inhibits VEGF (vascular endothelial growth factor-A). It has been approved as a second line brain tumor (gliomas) for recurrences. This agent has been used intra-arterially for localized prevention of stenosis.

Trastuzumab (Herceptin): is a monoclonal antibody that blocks the HER2/neu receptor. Its main use is to treat breast cancers. The main limitation to using Herceptin in cases of metastatic breast cancer to the brain is the blood brain barrier.

Any chemotherapeutic agent used in the body to treat lung, renal, GI, liver, skin, prostate, and other cancers may be used with the stent. All of these tumors have the potential of spreading to the brain and often chemotherapeutic options are limited due to the BBB. Because metastatic tumors (i.e., of other organ origin) to the brain are much more common when compared to primary tumors (anaplastic astrocytomas, glioblastomas), use of the presently disclosed stent and methods for such metastatic tumors would provide a benefit to a larger population of patients.

BBBDs may include, without limitation, Mannitol and other osmotic agents, phosphodiesterase-5 (PDE-5) inhibitors, and RMP-7 and other bradykinin agonists. Mannitol has been traditionally used as a BBBD; it is a complex sugar and relies on an osmotic disruption. PDES-inhibitors operate by a blockade of PDE-5 which leads in a buildup of cGMP in the cell. This buildup increases the permeability and decreases the vascular tone, resulting in increased permeability. RMP-7 and other bradykinin agonists modulate the junctions seen in brain endothelial cells at the BBB.

In another exemplary combination of drugs, a radiosensitizer is disposed on the inner or outer cylindrical portion 20, 30, and a radioisotope on the complementary inner or outer cylindrical portion 20, 30. Other combinations will be apparent to one having skill in the art in light of the present disclosure. Radiosensitizers are a category of drugs which increase the efficiency of the radiation on tumor cells. This is a broad category of drugs and include drugs that increase oxygen uptake (e.g., 5-Iododeoxyuridine (IUdR), 6-Bromodeoxyuridine (BUdR), Carbogen, Nicotinamide, Misonidazole, Metronidazole, 2-Deoxy-D-glucose (2DG), Lonidamine, Motexafin Gadolinium (MGd), Efaproxiral, RSR-13, Etanidazole, Pimonidazole, Trans Sodium Crocetinate), inhibit repair of DNA or stabilize DNA (e.g., Difluromethylornithine (DFMO), Poly(ADP-ribose) polymerase-1 (PARP-1) inhibitors, Heat Shock Protein-90 (HSP-90) inhibitors, Cell cycle checkpoint inhibitors, Cyclo-Oxygenase-2 (COX-2) inhibitors, Curcumin, Epothilone B), or any other mechanism where the tumor cell will be more susceptible to radiotherapy.

Brachytherapy is a term for radiotherapy where the radiation source is placed in or adjacent to a treatment target. Endovascular brachytherapy plays a small but notable role in medicine. In treatments of Liver cancer, spheres of a radioisotope are injected in an artery into the liver. Radioisotopes have been placed on stents to prevent restenosis at the immediate site of the stent. The radioactive nature of such stents inhibit restenosis by inhibiting nearby smooth muscle and endothelial cell proliferation as well as local inflammatory response. These radioisotopes include Irridium-192, Iodine-125, radium-226, radon-222, cobalt-60, cesium-137, gold-198, palladium-103, amongst many more. Some embodiments of the present disclosure are directed to placement of such radioisotopes on a DES in order to target a downstream lesion. As such, a radioisotope may be disposed on the inner and/or outer cylindrical portion 20, 30 of the stent 10 to serve as an effective therapy or adjunctive therapy.

In some embodiments, the outer cylindrical portion 20 is configured with low porosity thereby reducing drug delivery to the area underneath the stent 10. The inner cylindrical portion 30 may be configured with high porosity. And the interlumen space (between the interlumen surfaces 22, 34) functions as a secondary path for blood flow. The number and placement of cross-links 40 may be designed so as to minimize disruption to blood flow through this interlumen space. As blood flows through the stent 10, the drug eluted off the inner cylindrical portion 30 may be carried faster and/or further downstream than the drug on the outer cylindrical portion 20 due to a less disrupted flow through the inner cylindrical portion 30. This creates a unique opportunity for early arrival and enhanced delivery of the drug of the inner cylindrical portion 30 to the distal target. With such a design, local uptake of the drug (e.g., beneath the stent 10) will be reduced or eliminated.

A stent may be formed from a metallic weave. Such a weave creates a plurality of gaps (holes) defining a porosity of the stent. In known cardiac stents, high porosity (i.e., larger gaps) is used to facilitate diffusion of drug into the underlying endothelium. In flow-diverter stents, on the other hand, low porosity is used to promote flow through the stent, for example, bypassing an aneurysm. Porosity may be defined by percent material coverage, ranging from 0.1% to 99%. Some embodiments of the present disclosure are unique in utilizing a combination of a high porosity inner cylindrical portion 30 with a low porosity outer cylindrical portion 20. The outer cylindrical portion 30 may have a material coverage of, for example, 30-100%, thereby preventing drug diffusion to the area underneath the stent. The inner cylindrical portion 20, may have, for example, moderate material coverage of 10-30%.

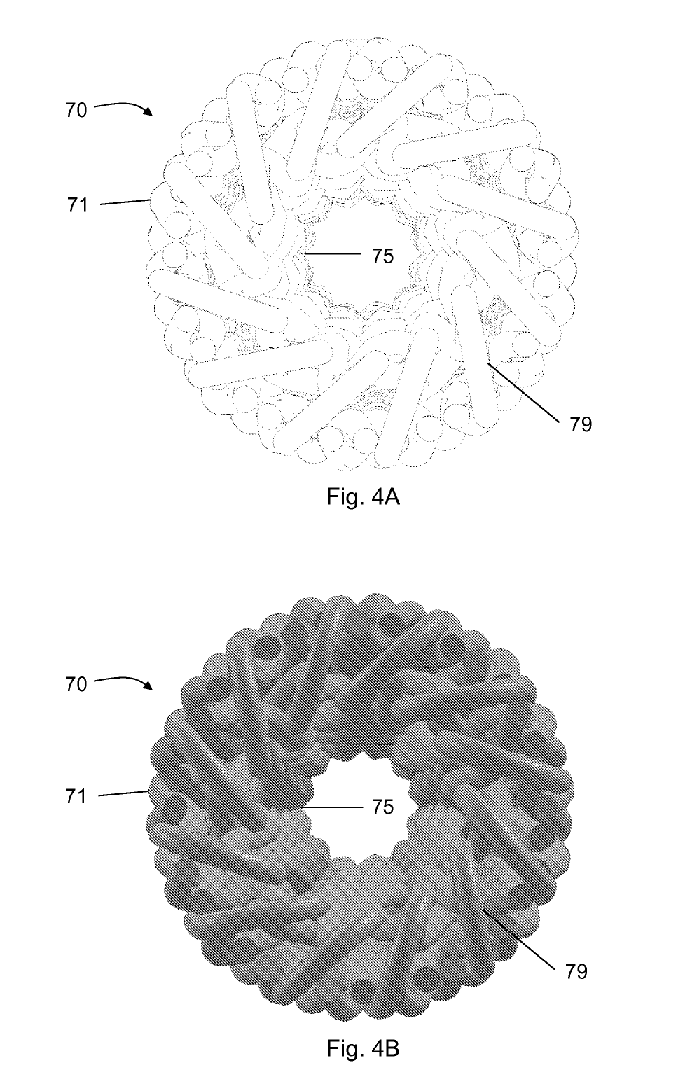

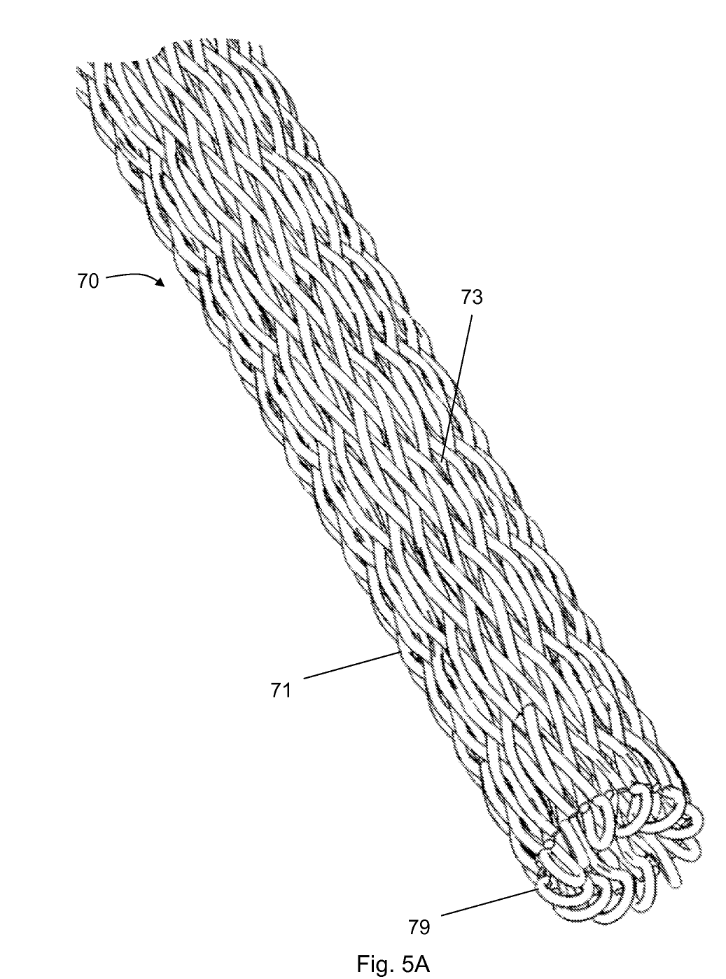

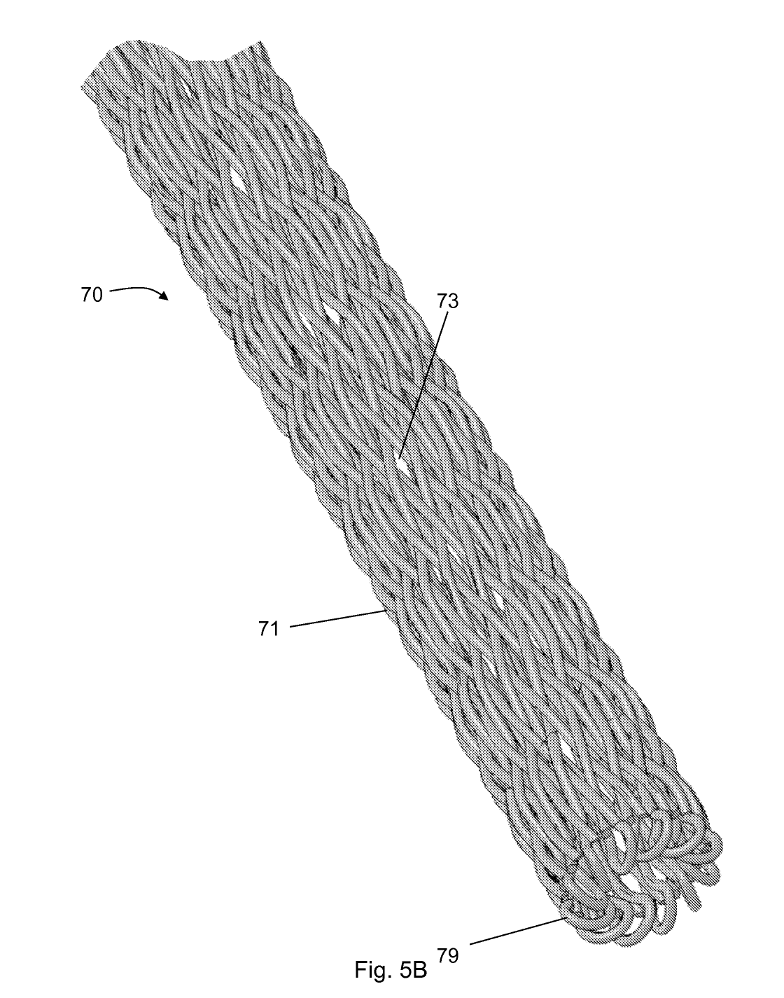

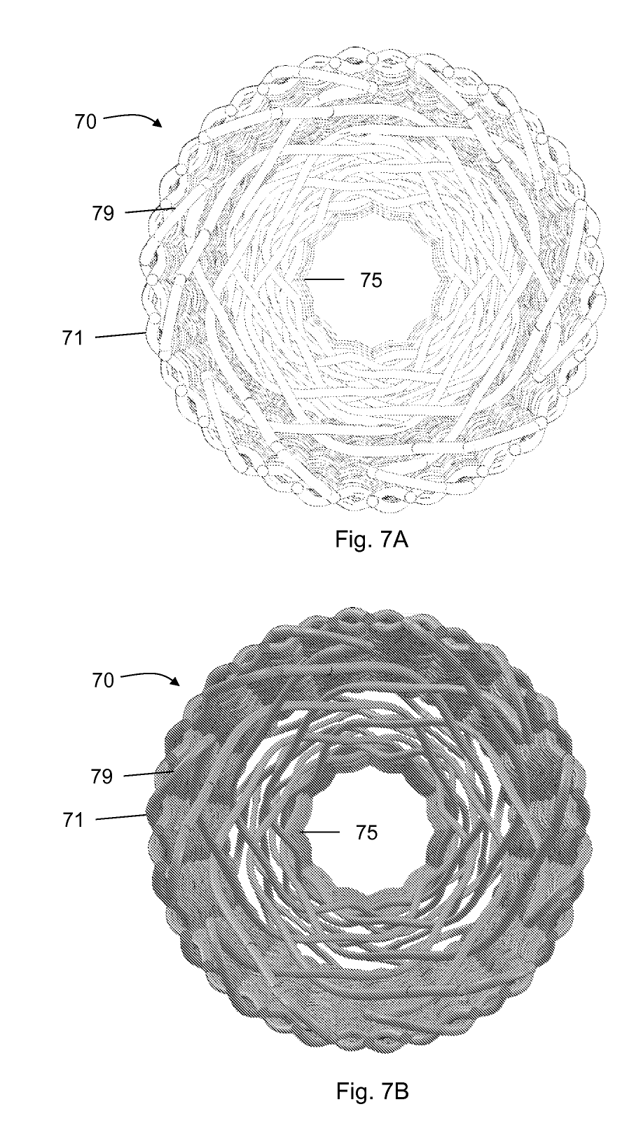

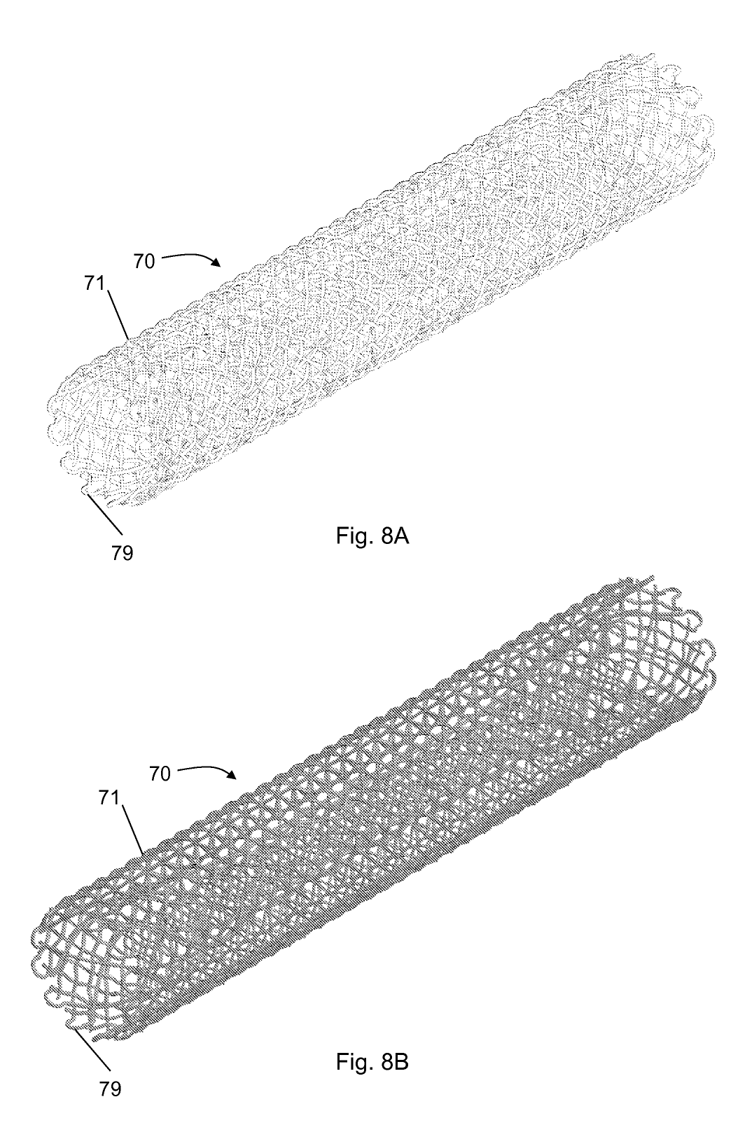

A stent formed from a metallic weave may also be described as a braided stent. FIGS. 4A-13B disclose one embodiment of a braided stent 70. In one embodiment, the stent 70 may have a closed configuration and an open configuration having different diameters (further described below). FIGS. 4A-6B depict the stent 70 in a closed configuration having an outer cylindrical portion 71. A plurality of openings 73 are disposed in the outer cylindrical portion 71. Each opening 73 extends through the outer cylindrical portion 71. The plurality of openings 73 define a porosity of the outer cylindrical portion 71. For example a greater number of openings 73, or openings 73 of a larger size, yield a higher porosity.

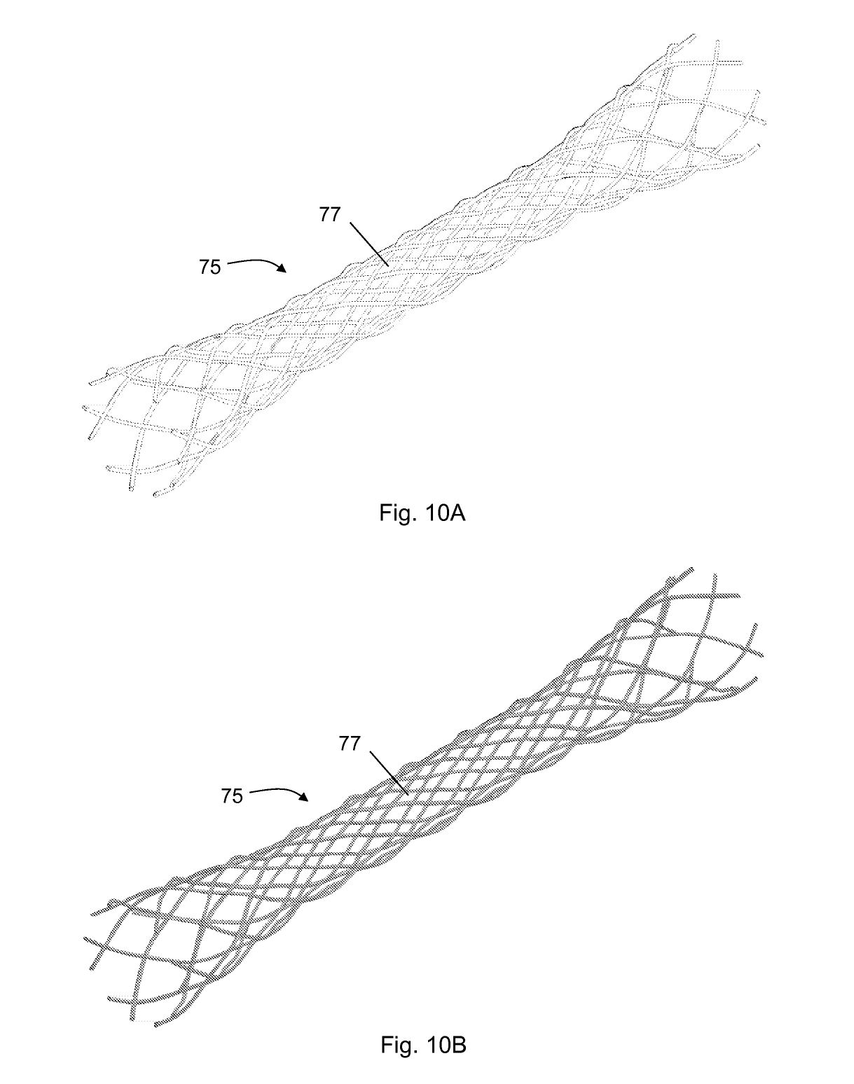

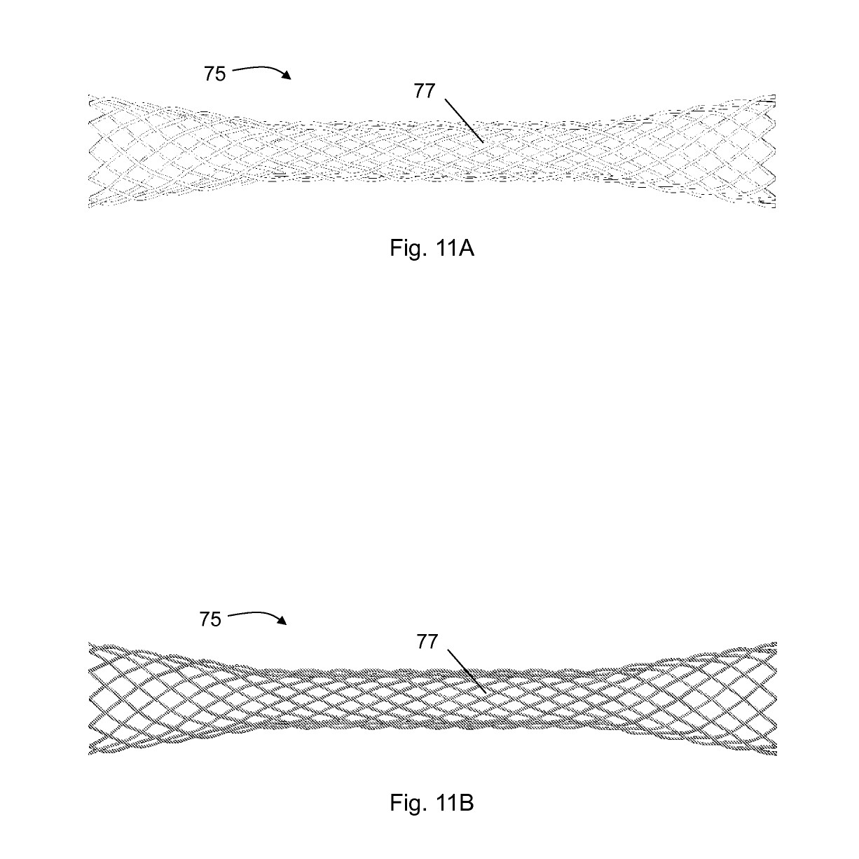

The stent 70 further comprises an inner cylindrical portion 75 disposed coaxially within the outer cylindrical portion 71. In FIGS. 10A-11B, the inner cylindrical portion 75 is shown independently of the stent 70. A plurality of openings 77 are disposed in the inner cylindrical portion 75. Each opening 77 extends through the inner cylindrical portion 75. The plurality of openings 77 define a porosity of the inner cylindrical portion 75, such that for example a greater number of openings 77, or openings 77 of a larger size, yield a higher porosity.

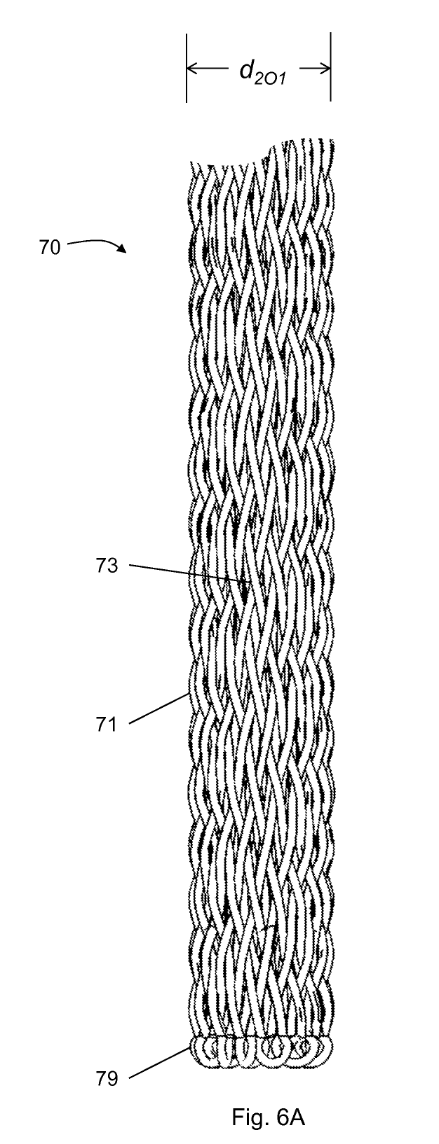

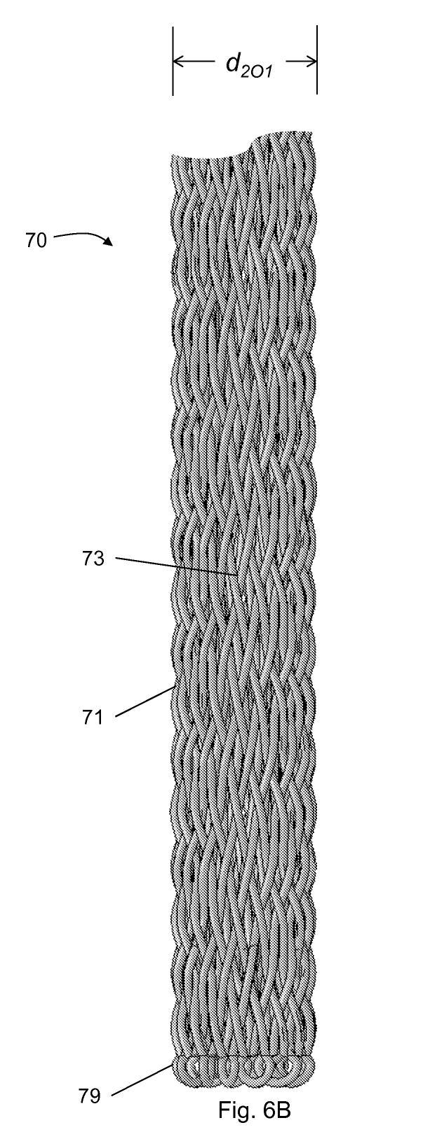

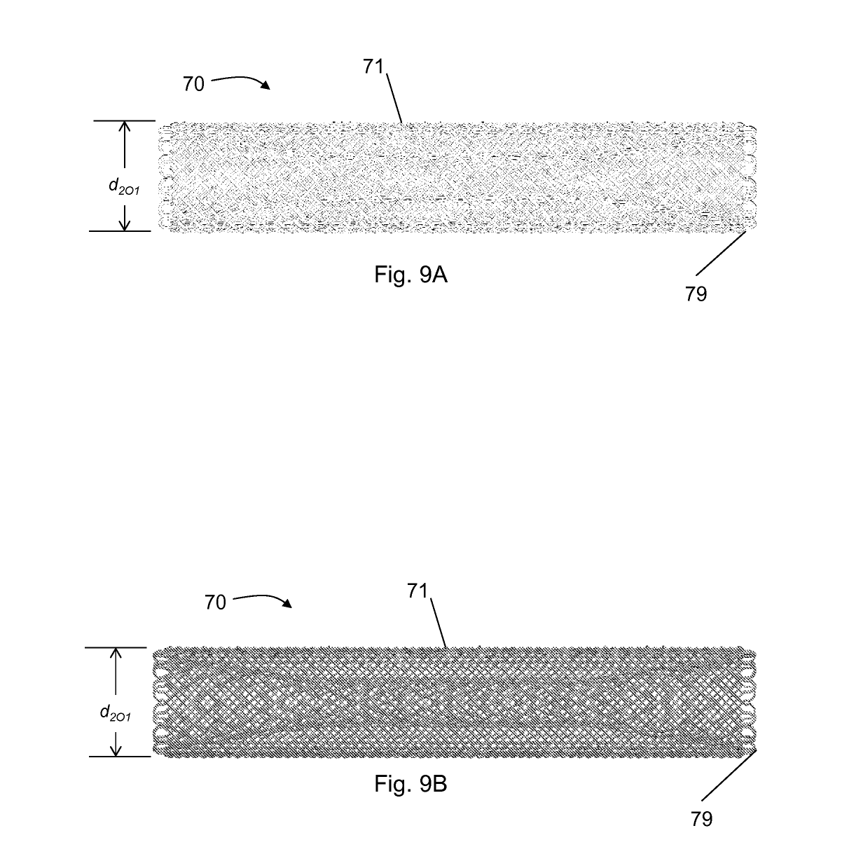

The stent 70 shown in FIGS. 4A-13B is configured to vary in diameter between the closed and open configurations. As such, the outer cylindrical portion 71 in the closed configuration is expandable from an outer first diameter d₂₀₁, for delivery into a vessel (see FIGS. 6A-6B), to an outer second diameter d₂₀₂, for placement at a location within the vessel (see the outer cylindrical portion 71 in the open configuration, Figures. 9A-9B). The inner cylindrical portion 75 may be configured to vary in diameter between the closed and open configurations. As such, the inner cylindrical portion 75 may be expandable from an inner first diameter, for delivery into a vessel, to an inner second diameter, for placement at a location within the vessel.

The stent 70 comprises a plurality of cross-links 79, which connect the outer cylindrical portion 71 to the inner cylindrical portion 75. The cross-links 79 are configured to allow a radial distance between the outer cylindrical portion 71 and the inner cylindrical portion 75 to vary. In this way, the cross-links 79 are considered to be collapsible (and expandable).

The braid or weave of the stent 70 may be configured to allow a precise determination of the stent design parameters, such as wire thickness and number of wires. In the open configuration (as shown in FIGS. 10A-11B), the inner cylindrical portion 75 may be sloped, parabolic, or any other configuration. As such, the diameter of the inner cylindrical portion 75 may vary over the length of the inner cylindrical portion 75. In one embodiment, the diameter of the inner cylindrical portion 75 is constant. The slope of the inner cylindrical portion 75, as well as the smallest diameter of the inner cylindrical portion 75 can be selected depending on a desired blood flow profile, the desired blood flow velocity needed to regulate drug elution, or other characteristics.

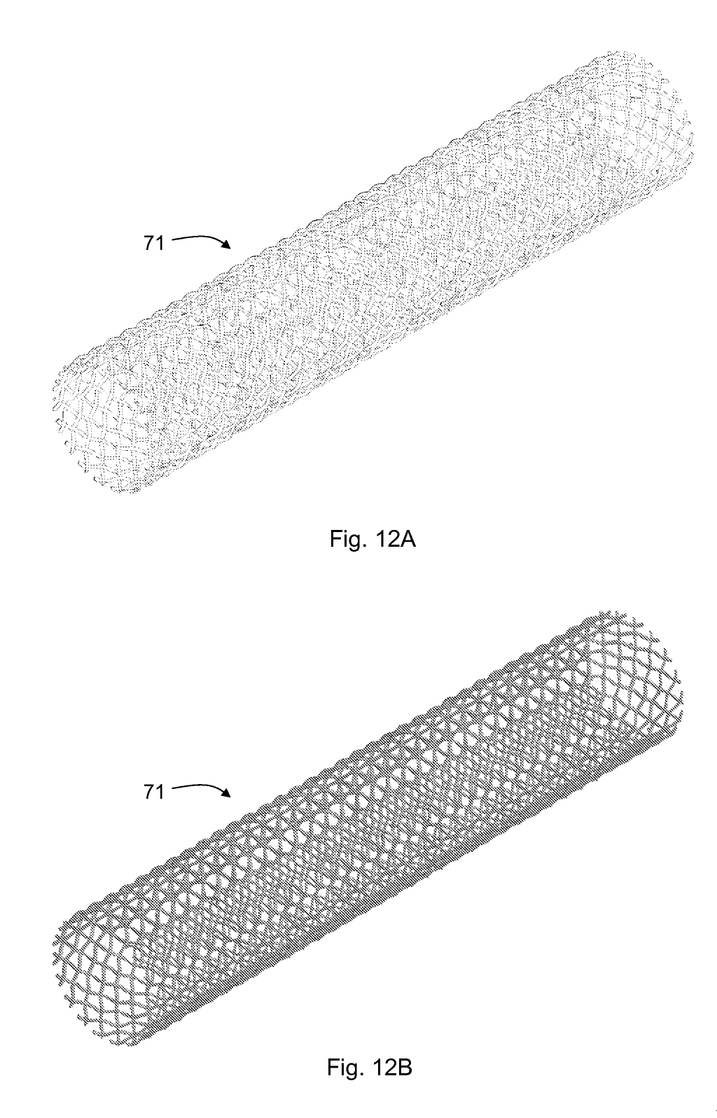

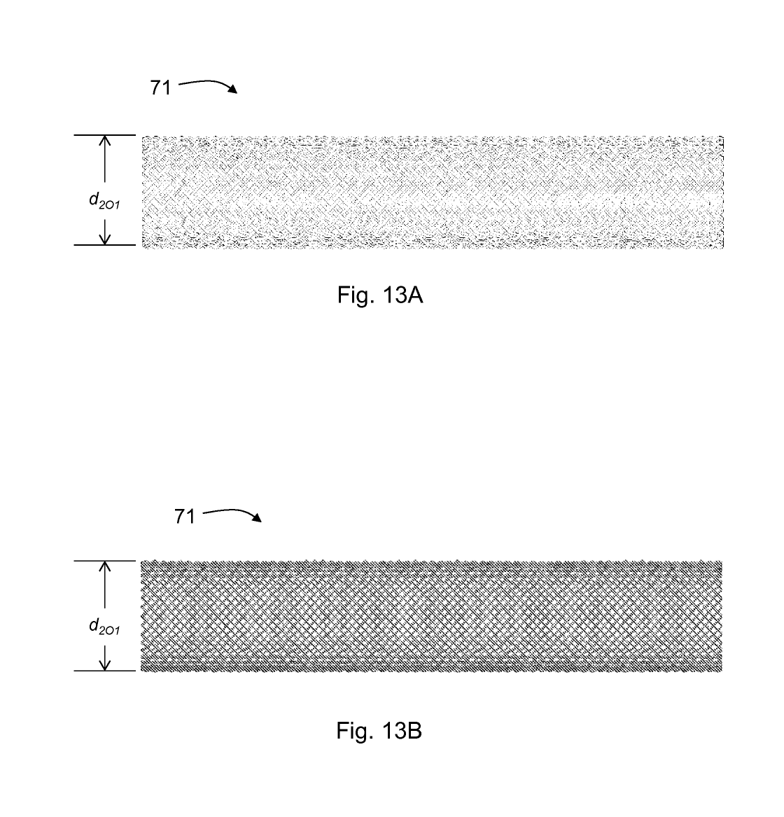

In one embodiment, the inner cylindrical portion 75 may be manufactured separately from the outer cylindrical portion 71 (as shown in FIGS. 12A-13B). The inner cylindrical portion 75 may later be cross-linked to the outer cylindrical portion 71. The portions 71, 75 may be cross-linked, for example, mechanically (i.e., braiding), by adhesives, or by welding. In another embodiment, the inner cylindrical portion 75, outer cylindrical portion 71, and cross-links may be formed at or near the same time. Different materials may be used for the inner cylindrical portion 75 and the outer cylindrical portion 71. For example, Nitinol may be used to form the inner cylindrical portion and platinum alloys or stainless steel maybe used for the outer cylindrical portion. In other embodiments, the materials may be the same.

The disclosed stents may be formed from any materials suitable for such stents, including metals such as, for example, cobalt chromium alloy (e.g., ELGILOY), stainless steel (e.g., 316L), high nitrogen stainless steel (e.g., BIODUR 108), cobalt chrome alloy (e.g., L-605), MP35N, MP20N, ELASTINITE (e.g., Nitinol), tantalum, nickel-titanium alloy, platinum-iridium alloy, gold, magnesium, or combinations. In other embodiments, bioabsorabable/biocompatible materials may be used, such as, for example, polylactide, poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly(alpha-hydroxy acid), DLPLA-poly(di-lactide), or combinations. Combinations of any suitable materials may be used. For example, the outer cylindrical portion 20, 71 may be formed from a biodegradable material with a metallic shell to prevent diffusion of the drug into the underlying segment of the blood vessel. In another example, the outer cylindrical portion 20, 71 is made from a metal and the inner cylindrical portion 30, 75 is made from a biodegradable material.

Previous stents require relatively high tensile strength materials due to their use with stenotic lesion (i.e., bracing open a stenotic portion of a blood vessel). For example, previous stents made use of materials like stainless steel, cobalt-chromium alloys, Nitinol, etc. which had high tensile strengths ranging from approximately 300-1200 MPa, inclusive. Therefore, previous stents could not make significant use of biodegradable/biocompatible materials (e.g., PLLA, PGA, etc.), which have low tensile strengths, ranging from approximately 20-300 MPa (or lower). However, the presently disclosed stent s are useful for treating non-stenotic lesions and, as such, can make extensive use of low and/or high strength materials.

The cross-links 40, 79 may be formed from any material suitable for such uses (as described above) including, for example, metals and biodegradable materials. In embodiments where low- strength materials are used of the inner and/or outer cylindrical portions (e.g., biodegradable materials), the use of metallic cross-links 40, 79 may advantageously assist in deployment by expanding the disclosed stents.

The present disclosure may be embodied as a method 100 of treating a region of interest in an individual using a drug-eluting stent. The region of interest may be, for example, a tumor. The method 100 comprises the step of deploying 103 the drug-eluting stent into a blood vessel of the individual to a location upstream (with respect to blood flow within the vessel) of the region of interest and near the region of interest. Such a location advantageously allows drug delivery to the region of interest while reducing the systemic delivery to the individual. The deployed stent is a dual-drug stent, for example, but not necessarily, a dual-drug stent as described above. In the upstream location, the drugs will be eluted into the bloodstream and carried to the region of interest. The drugs of the dual-drug stent are selected such that a first drug has the effect of increasing the efficacy of the second drug.

The method 100 may further comprise the step of infusing 106 the first drug to the region of interest. For example, a catheter may be used to infuse a drug to a location upstream and near the region of interest as is known in the art. The second drug may also be infused 109 to the region of interest using the catheter. In this manner, during a single minimally-invasive procedure, boluses of a first and second drug may be administered to the region of interest and a drug-eluting stent may be deployed for sustained drug delivery for days, weeks, or months after the procedure.

In a particular example, a catheter is navigated to the feeding artery of a tumor, and a BBBD is infused using the catheter. Next, a chemotherapeutic agent is infused. At this point, without changing the position of the catheter, a dual-drug stent can be deployed into the feeding artery without adding significant time to the procedure. This would result in a large initial bolus of drug delivery to the tumor and a sustained drug delivery from the stent of a long period of time, thereby increasing the individual's chances of survival.

The present disclosure may be embodied as a method 200 for deploying a drug-eluting stent for treating a region of interest of an individual. The method 200 comprises the step of delivering 203 the stent to a blood vessel of the individual at an upstream location near the region of interest. The stent is caused 206 to expand such that the stent remains at the upstream location. The stent may be caused 206 to expand by positive action of the operator, for example, by using a deployment tool to expand the catheter. In other embodiments, the stent may be configured such that it is biased to assume an expanded configuration when released from a deployment sheath. For example, the collapsible cross-links described above will aid in the recoil of the stent when placed in the vessel. As described above, even if the tensile modulus is low for a biodegradable stent, the cross-links will help in expansion of the stent. Previous stents are used for deployment in stenotic lesions. Therefore the recoil and radial strengths of the stents are necessarily large, which often limits design options. In usage according to the present disclosure, the deployment may be in a non-stenotic lesion. As such, the deployment can be accomplished by a low tensile modulus stent. Since a large radial force is not required, there is decreased risk associated with stent placement.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A drug-eluting stent, comprising:
an outer cylindrical portion being expandable from a first diameter for delivery into a vessel, to a second diameter for placement in the vessel, the outer cylindrical portion having an interlumen surface, an abluminal surface, and a plurality of openings extending from the interlumen surface to the abluminal surface, and the outer cylindrical portion having a first porosity;
an inner cylindrical portion disposed within and substantially coaxial with the outer cylindrical portion, the inner cylindrical portion having a luminal surface and an interlumen surface, and a plurality of openings extending from the luminal surface to the interlumen surface, and the inner cylindrical portion having a second porosity;
a plurality of cross-links, each cross-link connecting the outer cylindrical portion to the inner cylindrical portion, and each cross-link being collapsible such that the distance between the outer cylindrical portion and the inner cylindrical portion may vary; and
a first drug affixed to at least one of the outer cylindrical portion and inner cylindrical portion.

2. The drug-eluting stent of claim 1, wherein the inner cylindrical portion is configured to be expandable from an inner first diameter for delivery into a vessel, to an inner second diameter for placement in the vessel.

3. The drug-eluting stent of claim 1, wherein the first porosity is lower than the second porosity.

4. The drug-eluting stent of claim 1, wherein each cross-link comprises a first segment and a second segment, the first and second segments connected by a pivot.

5. The drug-eluting stent of claim 4, wherein the first segment is rotatable about the pivot relative to the second segment, and the pivot is biased such that the first and second segments form an angle 0 with each other when the cross-link is in a relaxed state.

6. The drug-eluting stent of claim 4, wherein the pivot is a coil.

7. The drug-eluting stent of claim 1, wherein at least a portion of the stent is made from a material having a tensile strength which is less than 300 MPa.

8. The drug-eluting stent of claim 1, wherein the first drug is affixed to at least one of the luminal surface of the inner cylindrical portion, the interlumen surface of the inner cylindrical portion, the interlumen surface of the outer cylindrical portion, the abluminal surface of the outer cylindrical portion, and the plurality of openings of the at least one of the outer cylindrical portion and inner cylindrical portion.

9. The drug-eluting stent of claim 1, wherein a first drug is affixed to the outer cylindrical portion and a second drug is affixed to the inner cylindrical portion.

10. The drug-eluting stent of claim 9, wherein the first drug is a chemotherapeutic agent and the second drug is a blood-brain-barrier disrupter.

11. The drug-eluting stent of claim 9, wherein the second drug is a radioisotope.

12. The drug-eluting stent of claim 1, wherein a second drug is disposed on the first drug.

13. The drug-eluting stent of claim 12, wherein the first and second drug are disposed on each other in at least two alternating layers.

14. A method of deploying a drug-eluting stent for treating a distal region of interest of an individual, comprising the steps of:
inserting the stent into a blood vessel of the individual to an upstream, distal location with respect to the region of interest;
causing the stent to expand such that the stent remains at the upstream location.

15. A method of treating a region of interest of an individual using a drug-eluting stent, the drug-eluting stent having two drugs thereon, the method comprising the steps of:
deploying the drug-eluting stent into a blood vessel of the individual to an upstream location near the region of interest; and
wherein the stent elutes a first drug and a second drug into the blood of the blood vessel such that the first drug reaches the region of interest before the second drug.

16. The method of claim 15, wherein the stent elutes the first drug into a fluid flow through a first lumen of the stent and the stent elutes the second drug into a fluid flow through a second lumen of the stent, and the stent is configured such that the fluid flow through the first lumen has a higher velocity than the fluid flow through the second lumen.

17. The method of claim 15, wherein the second lumen is an inter-lumen space between coaxially arranged cylindrical portions of the stent.

18. The method of claim 15, further comprising the steps of:
infusing, using a catheter, the first drug to the region of interest; and
infusing, using the catheter, the second drug to the region of interest.

19. The method of claim 15, wherein the first drug is a blood-brain-barrier disrupter and the second drug is a chemotherapeutic agent.

20. The method of claim 15, wherein the first drug is a radiosensitizer and the second drug is a radioisotope.
